(19) United States
(12) Patent Application Publication
(10) Pub. No.: US 2016/0287429 A1
(43) Pub. Date: Oct. 6, 2016

(54) NASAL RELIEF AND SNORING STOPPING DEVICE

(71) Applicant: Yen-Chang LIN, Nantou County (TW)

(72) Inventor: Yen-Chang LIN, Nantou County (TW)

(21) Appl. No.: 15/037,849

(22) PCT Filed: Dec. 23, 2014
(86) PCT No.: PCT/CN2014/094654

§ 371 Date: May 19, 2016

(30) Foreign Application Priority Data
Dec. 27, 2013 (CN) 201310753192.9

(51) Int. Cl. A61F 5/56

(52) U.S. Cl. CPC A61F 5/566

(57) ABSTRACT

An airflow intrusive nasal relief and snoring stopping device is applied in an oral cavity. The oral cavity includes an oral opening, an upper tooth, a tongue, a palate and a uvula. The nasal relief and snoring stopping device includes a tooth accommodating member accommodating the upper tooth, and an airflow intrusive member between the uvula and the oral opening The tooth accommodating member includes a left tooth region and a right tooth region that are symmetrical. The airflow intrusive member connects the left tooth region and the right tooth region, and extends from the tooth accommodating member towards the palate. With the airflow intrusive member intruding the airflow inhaled through oral opening, soft tissues in the oral cavity are prevented from the effect of the airflow and hence from vibrations that may cause snoring, thereby improving symptoms of nose congestion and allergies.

### FIELD OF THE INVENTION

The present invention relates to a snoring stopping device, and particularly to an nasal relief and snoring stopping device.

### BACKGROUND OF THE INVENTION

Snoring is a symptom of making a sound while asleep. A mild snoring symptom within a tolerable range usually does not need to seek for medical advice. However, a severe snoring symptom not only affects the sleeping quality of one and people around, but also may develop into “obstructive sleep apnea” that further jeopardizes the health.

There are numerous reasons causing snoring. One of the most common reasons is that, the muscles that originally stabilize and stretch the throat become lax during sleeping, such that the muscles in the oral cavity are receded to result in a narrowed respiratory tract. As the respiratory tract is narrowed, the wind speed of air inhaled becomes greater, causing the soft palate, uvula, tongue and tonsils in a lax state to vibrate and to make a sound.

There are three common treatments for the snoring symptom: 1) continuous positive airway pressure (CAPA), which directly injects air through a breathing mask; 2) uvulopalatopharyngoplasty (UPPP), which removes the soft palate and uvula by a surgery to reduce the obstruction effect of the tongue; and 3) a mandibular advancement device (MAD), which uses a corrector made by a dental method to hold the lower jaw to bite at a position stretched forward. As the tongue muscles are mainly connected to the front inner side of the mandible, the tongue is pulled upwards when the mandible bites forward during sleeping. Thus, by reducing the blockage of the respiratory tract to allow unobstructed breathing, the snoring is naturally alleviated or completely eliminated. For example, the U.S. Pat. No. 6,055,986, “Apparatus and Method for the Reduction of Snoring”, discloses a device for stopping snoring. The above disclosure includes an upper curved platform, a lower curved platform, and a connecting material bonded to the upper and lower curved platform. The upper curved platform and the lower curved platform are adapted to receive the bite of a user, and the lower curved platform is steadily connected to the upper curved platform using the connecting material. Thus, the muscles at the throat do not become lax and recede to block the respiratory tract during sleeping to accordingly improve the snoring. Although using such snoring stopping device may alleviate or even completely cure the snoring symptom, because the snoring stopping device is held in the oral cavity during the period of sleeping, the user may feel discomforts or even sore muscles between the teeth and the mandibular joints.

Further, snoring is closely correlated to nose allergies. Nose allergies frequently cause swelling of nasal polyps, nasal meatus blockage, and a runny noise. As the nose becomes congested, the patient switches to breathe through the mouth. Breathing without the nose for an extended period of time, the patient may unconsciously develop a habit of breathing through the mouth, and it is then improbable for the nose congestion to improve. Under such vicious cycle, the issues of nose allergies and congestion continue to aggravate and become unsolvable.

### SUMMARY OF THE INVENTION

Therefore, it is a primary object of the present invention to improve the snoring condition occurring during sleeping.

It is another object of the present invention to solve discomforts caused during using a conventional snoring stopping device as well as the issue of sore muscles between teeth and mandibular joints caused after using such conventional snoring stopping device.

It is yet another object of the present invention to solve conditions of nose congestion and allergies.

To achieve the above objects, the present invention provides an nasal relief and snoring stopping device applied in an oral cavity. The oral cavity includes an oral opening, an upper tooth, a tongue, a palate located at one side of the upper tooth away from the tongue, and a uvula connected to one side of the palate away from the oral opening The nasal relief and snoring stopping device includes a tooth accommodating member accommodating the upper tooth, and an airflow intrusive member disposed between the uvula and the oral opening The tooth accommodating member includes a left tooth region and a left tooth region that are symmetrically disposed. The airflow intrusive member connects the left tooth region and the right tooth region, and extends from the tooth accommodating member towards the palate.

It is known from the above description that, the present invention provides following features.

1. With the airflow intrusive member intruding the airflow inhaled through the oral cavity, soft tissues in the oral cavity are not vibrated by the blowing of the airflow, so that snoring conditions of a user are improved.

2. As the airflow intrusive member disposed reduces the ratio of the airflow inhaled through the oral cavity, the user is forced to breathe through the nose. Thus, by continually using the nasal relief and snoring stopping device, the condition of the user breathing through the oral cavity during sleeping may be corrected to allow the user to develop the habit of breathing through the nose. By forcing to breathe through the nose, human self healing abilities can be triggered to improve symptoms of nose congestion and allergies.

### BRIEF DESCRIPTION OF THE DRAWINGS

FIG. 1 is a perspective structural diagram of the present invention;

FIG. 2 is a rear schematic diagram of the present invention;

FIG. 3 is a schematic diagram of a first embodiment of the present invention worn in a normal state;

FIG. 4 is a schematic diagram of the first embodiment of the present invention worn in a sleeping state;

FIG. 5 is a schematic diagram of a second embodiment of the present invention worn in a sleeping state;

FIG. 6 is a schematic diagram of a third embodiment of the present invention worn in a sleeping state;

FIG. 7 is a rear schematic diagram of a fourth embodiment of the present invention;

FIG. 8 is a rear schematic diagram of a fifth embodiment of the present invention;

FIG. 9 is a schematic diagram of a sixth embodiment of the present invention worn in a sleeping state;

FIG. 10 is a schematic diagram of a seventh embodiment of the present invention worn in a sleeping state; and

FIG. 11 is a schematic diagram of an eighth embodiment of the present invention worn in a sleeping state.

### DETAILED DESCRIPTION OF THE PREFERRED EMBODIMENTS

Details and technical contents of the present invention are given with the accompanying drawings below.

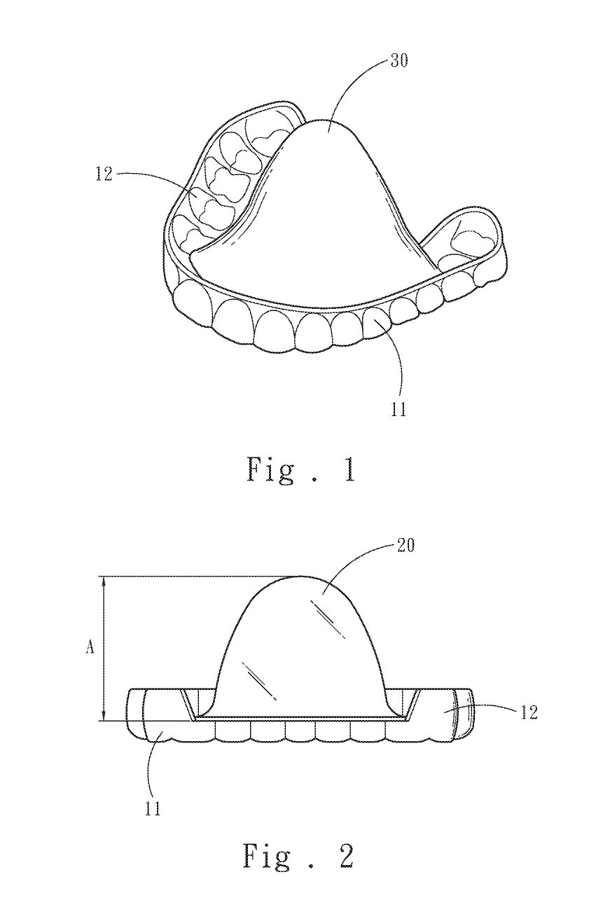

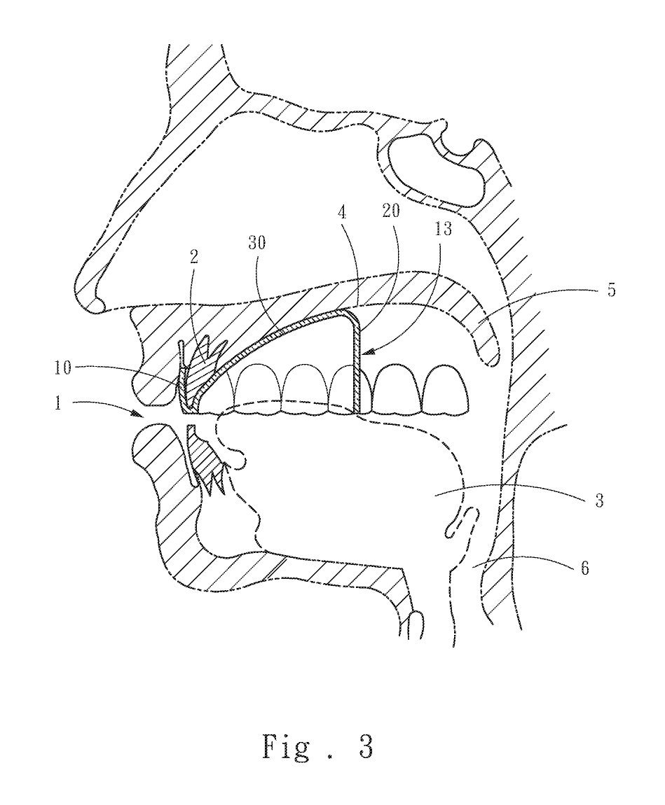

Referring to FIG. 1, FIG. 2 and FIG. 3, the present invention provides an nasal relief and snoring stopping device applied in an oral cavity. The oral cavity includes an oral opening 1, an upper tooth 2, a tongue 3, a palate 4 located at one side of the upper tooth 2 away from the tongue 3, and a uvula 5 connected to one side of the palate 4 away from the oral opening 1. The nasal relief and snoring stopping device includes a tooth accommodating member 10 accommodating the upper tooth 2, and an airflow intrusive member 20 disposed between the uvula 5 and the oral opening 1. The tooth accommodating member 10 includes a left tooth region 11 and a right tooth region 12 that are symmetrically disposed. The airflow intrusive member 20 connects the left tooth region 11 and the right tooth region 12, and extends from the tooth accommodating member 10 towards the palate 4 to abut against the palate 4. By intruding the airflow inhaled through the oral opening 1 with the airflow intrusive member 20, not only the airflow speed of the airflow is reduced, but also the airflow is prevented from coining into direct contact with soft tissues in the oral cavity after entering the oral cavity and thus from vibrating the soft tissues. The tooth accommodating member 10 and the airflow intrusive member 20 may be made of a material selected from rubber, resin and plastic.

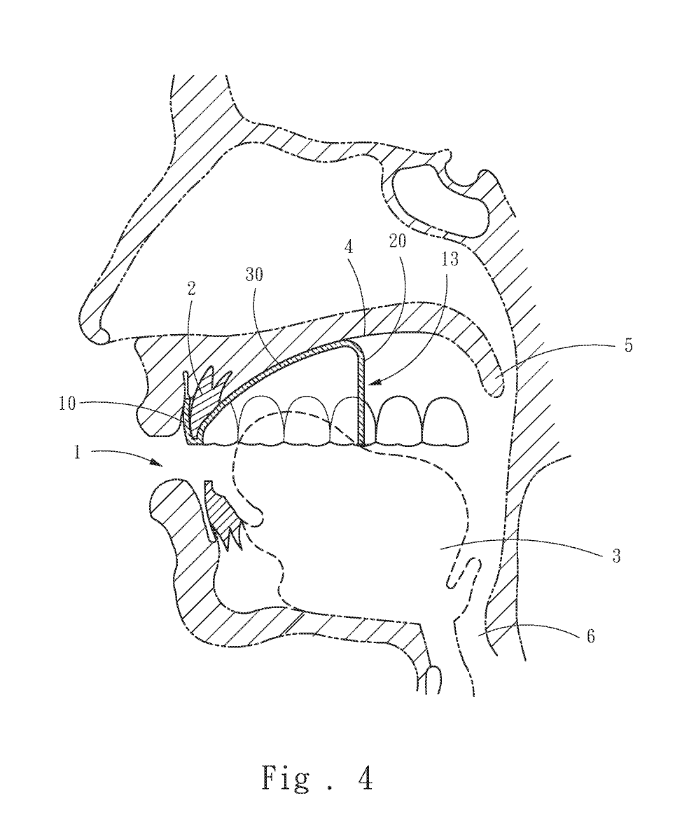

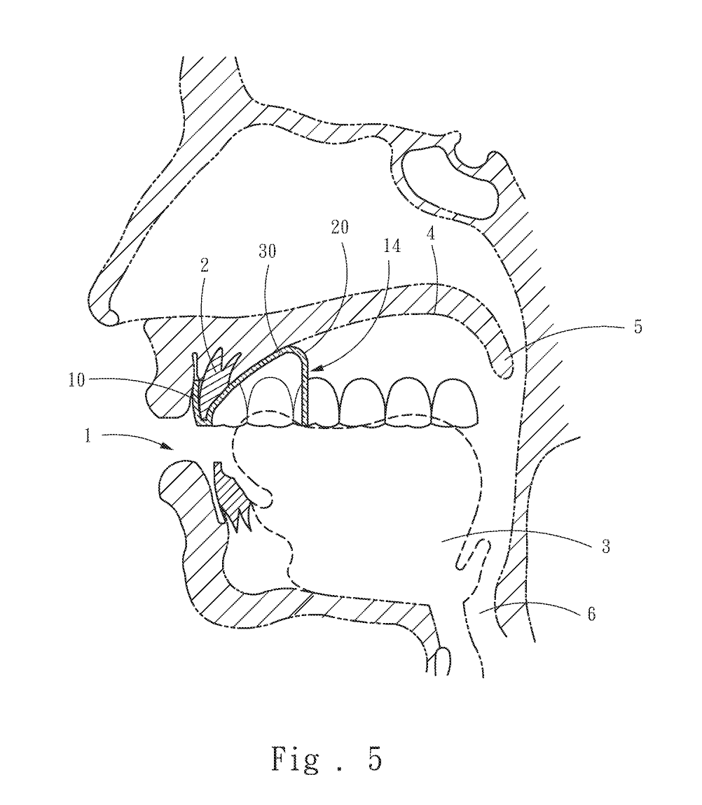

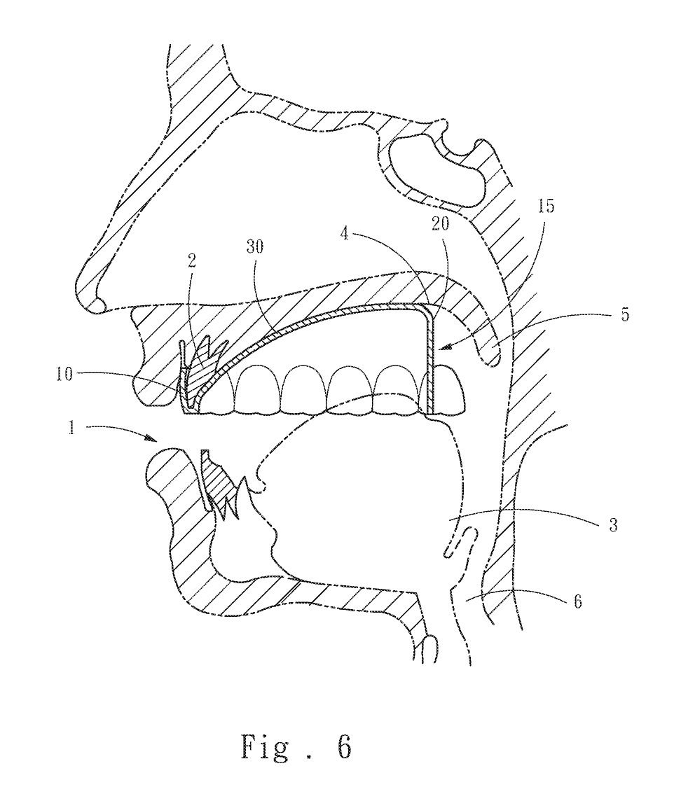

The present invention is capable of effectively reducing issues of snoring or sleep apnea caused by blockage of a throat 6 due to the tongue 3 that is excessively receded. Referring to FIG. 3, FIG. 4, FIG. 5 and FIG. 6 showing schematic diagrams according to different embodiments of the present invention, the present invention further includes an abutting member 30, which extends from an edge of the airflow intrusive member 20 along the palate 4 towards away from the uvula 5 to become connected to the tooth accommodating member 10. Further, the abutting member 30, the tooth accommodating member 10 and the airflow intrusive member 20 are a formed integral. Referring to FIG. 3 showing a first embodiment of the present invention, the airflow intrusive member 20 is disposed at a first wind blocking position 13. When a user is not yet in a sleeping state, muscles in the oral cavity are at normal positions, and the airflow intrusive member 20 is abutted on the tongue 3. Referring to FIG. 4, when the user is in a sleeping state, the tongue 3 is receded as the muscles in the oral cavity become lax. Thus, with the airflow intrusive member 20 disposed, the recession level of the tongue 3 is limited by the airflow intrusive member 20, thereby preventing snoring and solving the issue of sleep apnea.

Further, with the airflow intrusive member 20 disposed, the ratio of airflow inhaled through the oral cavity is reduced to force the user to breathe through the nose, hence correcting the habit of unconsciously breathing through the oral cavity during sleeping. By forcing to breathe through the nose, human self healing abilities can be triggered to improve symptoms of nose congestion and allergies, and to solve dryness and discomforts in the oral cavity caused by breathing through the oral cavity during sleeping.

Referring to FIG. 5 and FIG. 6 showing a second embodiment and a third embodiment of the present invention in application, the position of the airflow intrusive member 20 may be differently selected according to different user requirements when the nasal relief and snoring stopping device is manufactured. In the second embodiment, as shown in FIG. 5, the airflow intrusive member 20 is disposed at a second wind blocking position 14, which is between the first wind blocking position 13 and the oral opening 1. Since the second wind blocking position 14 is farther away from the uvula 5 than the first wind blocking position 13, discomforts caused from using the nasal relief and snoring stopping device can be alleviated. In the third embodiment, as shown in FIG. 6, the airflow intrusive member 20 is disposed at a third wind blocking position 15, which is between the first wind blocking position 13 and the uvula 5. Since the third wind blocking position 15 is closer to the uvula 5 than the first wind blocking position 13, the intruding effect of the airflow intrusive member 20 upon the airflow can be reinforced to reduce the effect that the airflow has on the soft tissues in the oral cavity and to further reduce the frequency of snoring. As the user gradually gets used to wearing the nasal relief and snoring stopping device of the present invention, the airflow intrusive member may be progressively adjusted according to an order from the second wind blocking position, the first wind blocking position to the third wind blocking position.

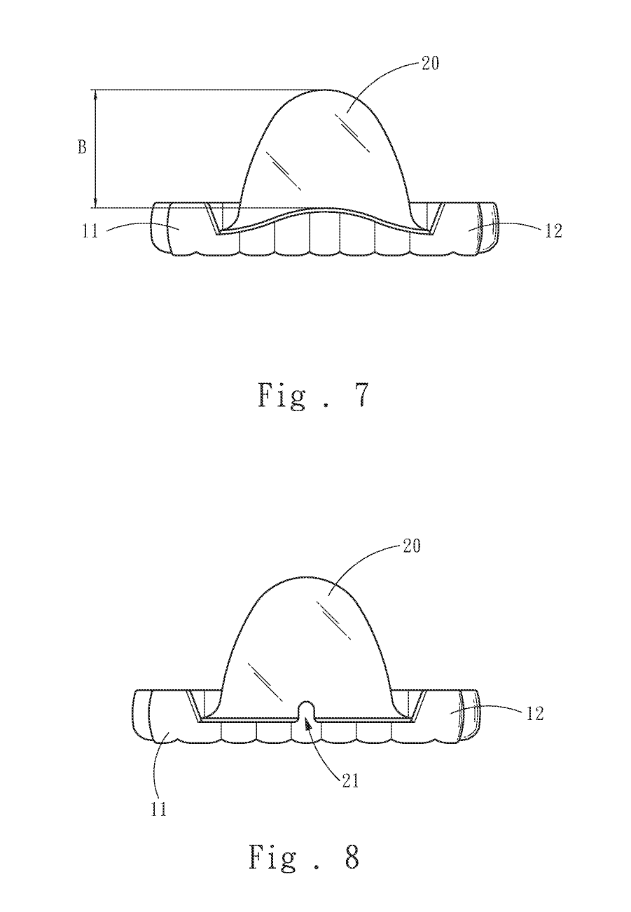

Referring to FIG. 2 and FIG. 7, in the first embodiment of the present invention, the airflow intrusive member 20 has a first height A. Thus, when the user is in a sleeping state, the airflow intrusive member 20 may be abutted at the tongue 3 to prevent the tongue from 3 blocking the throat 6 due to lax muscles, as shown in FIG. 2. However, according to different user habits, the present invention further provides a fourth embodiment, as shown in FIG. 7. In the fourth embodiment, the airflow intrusive member 20 has a second height B smaller than the first height A, such that the airflow intrusive member 20 is kept away from the tongue 3 to reduce discomforts caused from using the nasal relief and snoring stopping device.

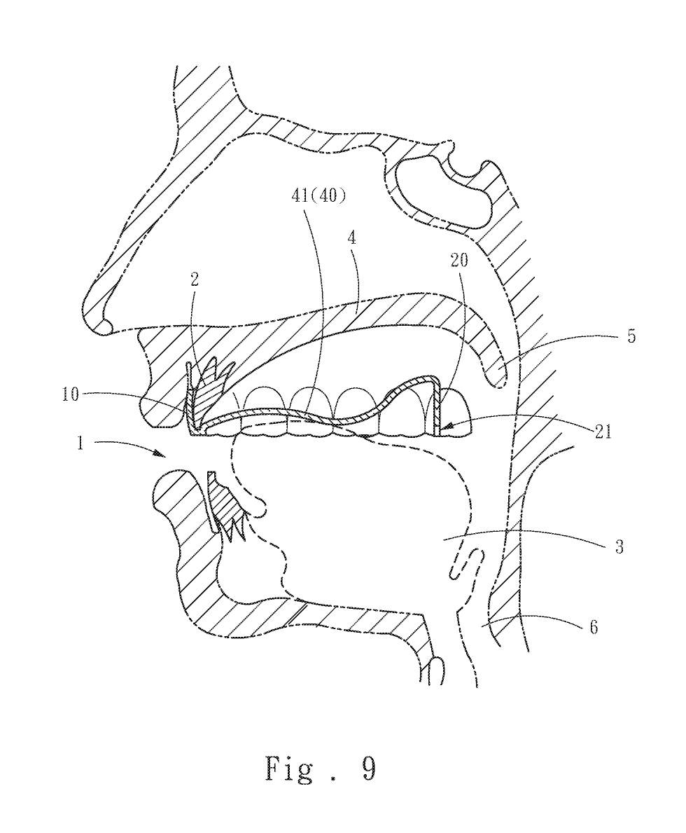

Referring to FIG. 8, to prevent accumulated saliva naturally produced in the oral cavity from causing discomforts, in a fifth embodiment of the present invention, the airflow intrusive member 20 further includes a liquid discharging portion 21 disposed at one end away from the palate 4. Through the liquid discharging portion 21, the saliva in the oral cavity may flow into the throat 6 and be swallowed to solve the issue of accumulated saliva in the oral cavity.

To further enhance effects of the nasal relief and snoring stopping device for improving snoring or sleep apnea, a sixth embodiment of the present invention further includes a tongue pressing member 40. Referring to FIG. 9, the tongue pressing member 40 includes a pressing portion 41. The pressing portion 41 extends from an edge of the airflow intrusive member 20 towards away from the uvula 5, is connected to the tooth accommodating member 10, and protrudes towards away from the palate 4. As such, when the tongue 3 is receded due to lax muscles in the oral cavity, the tongue 3 is prevented from further receding through the pressing portion 41, so as to further reduce the recession level of the tongue 3. In other words, the pressing portion 41 of the tongue pressing member 40 increases the contact area between the nasal relief and snoring stopping device and the tongue 3, hence further reinforcing the resisting abilities of the tongue 3 against recession.

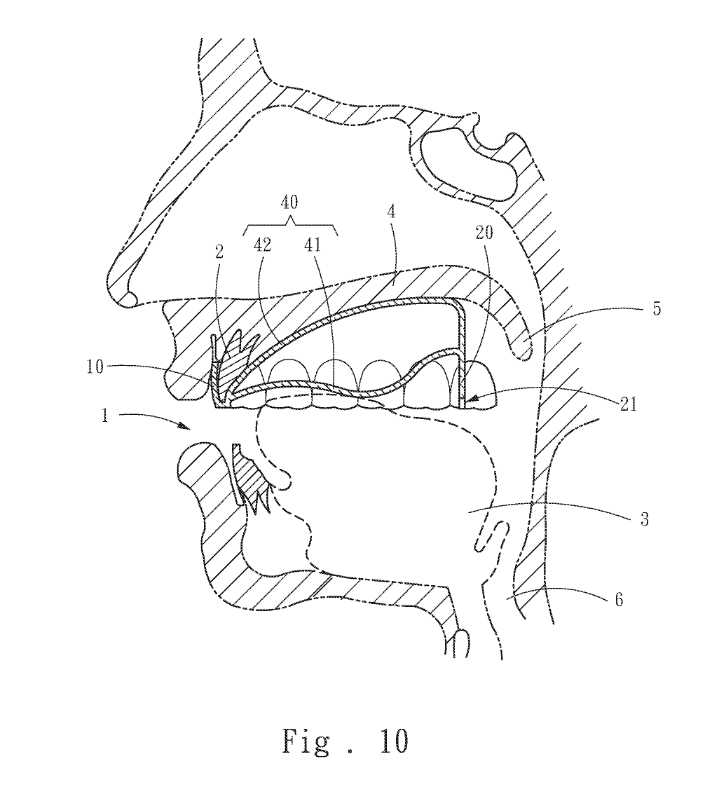

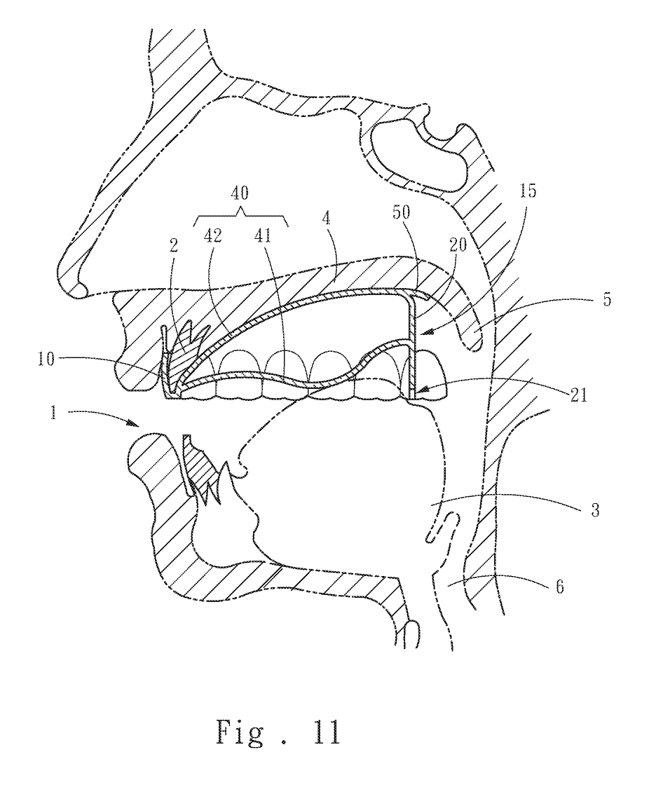

Referring to FIG. 10 showing a seventh embodiment of the present invention, the tongue pressing member 40 further includes a support portion 42 disposed at one side of the pressing portion 41 near the palate 4. In the embodiment, the support portion 42 abuts against the palate 4, and between the pressing portion 41 and the support portion 42 is a hollow design. It should be noted that, the non-limiting embodiments described above are for better understanding the present invention, and the present invention is not limited to these details in actual applications. For example, the height of the support portion 42 is determined according to the height of the airflow intrusive member 20, and is not limited to abutting against the palate 4 as described in the embodiment. Further, between the pressing portion 41 and the support portion 42 may be a solid design, and may be selected according to user habits.

Referring to FIG. 11, an eighth embodiment of the present invention further includes an extension member 50. The extension member 50 is disposed at one end of the airflow intrusive member 20 near the palate 4, and extends from the airflow intrusive member 20 towards the uvula 5 to be adhered to the palate 4. Thus, the extension member 50 is abutted against the root of the uvula 5, so as to prevent snoring caused by the uvula 5 due to lax muscles in the oral cavity during sleeping. As the human body possesses good self healing abilities and environment resisting abilities, such human self healing abilities can be triggered by appropriately forcing to breathe through the nose in the situation of nose congestion. Based on human tests of dozens of users, the nasal relief and snoring stopping device not only provides the effect of improving snoring but also solves the issue of nose congestion and allergies. The above effects get more noticeable over an extended period of use.

In conclusion, the present invention provides following features.

1. With the airflow intrusive member that intrudes the airflow inhaled through the cavity opening, the soft tissues in the oral cavity are not vibrated by the blowing of the airflow, hence improving the snoring condition of the user.

2. Through replacing a biting design of upper and lower jaws as used by a conventional snoring stopping device by the airflow intrusive method, the present invention prevents discomforts caused during using the device in the oral cavity and sore muscles caused after using the device.

3. The airflow intrusive member is abutted against the tongue, and prevents the issue of the tongue blocking the throat due to lax muscles during sleep, hence improving conditions of snoring and sleep apnea.

4. The airflow intrusive member is stabilized using the abutting member, such that the airflow intrusive member is steadily abutted against the palate.

5. The position of the airflow intrusive member is adjustable according to application requirements, so as to enhance the effect of stopping snoring or reducing discomforts caused during the use.

6. By adjusting the height of the airflow intrusive member, discomforts caused during the use can be reduced.

7. The airflow intrusive member disposed reduces the ratio of airflow inhaled through the oral cavity to force the user to breathe though the nose. Thus, the continual use of the nasal relief and snoring stopping device is capable of correcting the condition of the user breathing through the oral cavity during sleep, and developing the habit of breathing through the nose for the user. By forcing to breathe through the nose, human self healing abilities can be triggered to improve symptoms of nose congestion and allergies, and to solve dryness and discomforts in the oral cavity caused by breathing through the oral cavity during sleeping.

8. With the liquid discharging portion disposed, the saliva in the oral cavity may flow through the liquid discharging portion into the throat and be swallowed, thereby solving the issue of accumulated saliva in the oral cavity and to enhance the ease of using the nasal relief and snoring stopping device.

9. By increasing the contact area between the pressing portion and the tongue, the tongue can be prevented from receding further due to lax muscles in the oral cavity during sleeping, thereby further enhancing the snoring stopping effect of the nasal relief and snoring stopping device.

10. The support portion is capable of reinforcing the structural strength of the tongue pressing member, so as to prevent deformation during the use of the nasal relief and snoring stopping device.

11. As the extension member is abutted against the root of the uvula, the uvula is prevented from not only drooping due to lax muscles in the oral cavity during sleeping but also vibrations due to the effect of the airflow, thereby further improving the snoring condition.

What is claimed is:

1. An nasal relief and snoring stopping device, applied in an oral cavity, the oral cavity comprising an oral opening, an upper tooth, a tongue, a palate located at one side of the upper tooth away from the tongue, and a uvula connected to one side of the palate away from the oral opening, the nasal relief and snoring stopping device comprising:
a tooth accommodating member, accommodating the upper tooth, comprising a left tooth region and a right tooth region that are symmetrically disposed; and
an airflow intrusive member, disposed between the uvula and the oral opening, connecting the left tooth region and the right tooth region, extending from the tooth accommodating member towards the palate.

2. The nasal relief and snoring stopping device of claim 1, further comprising:
an abutting member, extending from an edge of the airflow intrusive member along the palate towards away from the uvula, and connected to the tooth accommodating member.

3. The nasal relief and snoring stopping device of claim 1, wherein the tooth accommodating member further comprises a first wind blocking position, a second wind blocking position between the first wind blocking position and the oral opening, and a third wind blocking position between the first wind blocking position and the uvula, and the airflow intrusive member is disposed at the first wind blocking position.

4. The nasal relief and snoring stopping device of claim 1, wherein the tooth accommodating member further comprises a first wind blocking position, a second wind blocking position between the first wind blocking position and the oral opening, and a third wind blocking position between the first wind blocking position and the uvula, and the airflow intrusive member is disposed at the second wind blocking position.

5. The nasal relief and snoring stopping device of claim 1, wherein the tooth accommodating member further comprises a first wind blocking position, a second wind blocking position between the first wind blocking position and the oral opening, and a third wind blocking position between the first wind blocking position and the uvula, and the airflow intrusive member is disposed at the third wind blocking position.

6. The nasal relief and snoring stopping device of claim 1, wherein the tooth accommodating member is made of a material selected from the group consisting of rubber, resin and plastic.

7. The nasal relief and snoring stopping device of claim 1, further comprising a tongue pressing member, the tongue pressing member comprises a pressing portion, the pressing portion extends from an edge of the airflow intrusive member towards away from the uvula, is connected to the tooth accommodating member, and protrudes towards away from the palate.

8. The nasal relief and snoring stopping device of claim 7, wherein the tongue pressing member further comprises a support portion that is disposed at one side of the pressing portion near the palate.

9. The nasal relief and snoring stopping device of claim 1, further comprising an extension member, which is disposed at one end of the airflow intrusive member near the palate, extends from the airflow intrusive member towards near the uvula, and is adhered to the palate.

10. The nasal relief and snoring stopping device of claim 1, wherein the airflow intrusive member further comprises a liquid discharging portion disposed at one end away from the palate.
